(19) United States
(12) Patent Application Publication
(10) Pub. No.: US 2020/0197544 A1
(43) Pub. Date: Jun. 25, 2020

(54) JCV IMAGING METHODS AND COMPOSITIONS

(71) Applicant: BIOGEN MA Inc., Cambridge, MA (US)

(72) Inventors: Ellen CAHIR-MCFARLAND, Cambridge, MA (US); Daniel BRADLEY, Cambridge, MA (US); Maciej KALISZCZAK, Arlington, MA (US)

(21) Appl. No.: 16/608,792

(22) PCT Filed: Apr. 26, 2017
(86) PCT No.: PCT/US2018/029538

§ 371 Date: Oct. 25, 2019

Related U.S. Application Data
(60) Provisional application No. 62/490,361, filed on Apr. 26, 2017.

(51) Int. Cl. A61K 51/04; C12Q 1/70; G01N 33/569

(52) U.S. Cl. CPC A61K 51/0491; C12Q 1/70; A61K 2123/00; G01N 2333/01; G01N 33/56983

(57) ABSTRACT

Provided herein, in some aspects, are methods of administering to a subject a thymidine kinase substrate having a label and detecting presence or absence of the label as an indication of the presence of a polyomavirus (e.g., JCV and BK).

### RELATED APPLICATION

This application claims the benefit under 35 U.S.C. § 119(e) of U.S. provisional application No. 62/490,361, filed Apr. 26, 2017, which is incorporated by reference herein in its entirety.

### BACKGROUND

Polyomaviruses are small, non-enveloped DNA viruses, which infect a large percentage of the population. With few exceptions, illnesses associated with these viruses occur in immunocompromised patients. The human polyomaviruses BK virus (BKV) and John Cunningham virus (JCV) are known to cause, respectively, hemorrhagic cystitis in recipients of bone marrow transplantation and progressive multifocal leukoencephalopathy (PML) in immunocompromised patients. JCV, in particular, can cross the blood-brain barrier into the central nervous system (CNS), where it infects oligodendrocytes and astrocytes resulting in multiple areas of demyelination.

### SUMMARY

Routine methods for determining whether a subject has a polyomavirus include detecting viral DNA or protein in a urine sample, a cerebrospinal fluid sample, or a blood sample from the subject. While these conventional methods of detecting polyomavirus are useful for identifying the presence or absence of the virus in a subject, they are not particularly useful for identifying anatomically-distinct viral reservoirs, which can be used to assess the degree of risk of a subject for developing a disease associated with certain polyomaviruses.

The experimental data provided herein demonstrates that thymidine kinase 1 (TK1) expression at the mRNA level and protein level is increased in human cells and brain tissue infected with JCV.

Thus, provided herein, in some embodiments, are methods for detecting the presence of a polyomavirus (e.g., JCV or BKV) in a subject by detecting higher than expected levels of a thymidine kinase (e.g., TK1) in at least one tissue of a subject.

In some embodiments, a method of detecting a polyomavirus (e.g., JCV) in at least one tissue of a subject comprises administering to the subject a thymidine kinase substrate having a label and detecting presence or absence of the label in at least one tissue of the subject, wherein presence of the label above a reference level is indicative of presence of a polyomavirus in the at least one tissue of the subject.

In some embodiments, the method of detecting a polyomavirus (e.g., JCV) in a subject comprises performing at least one assay to detect the presence or absence of the polyomavirus (e.g., JCV) in a subject and administering to the subject a thymidine analog having a radiolabel (e.g., 3′-deoxy-3′-[¹⁸F]-fluorothymidine (FLT)) and performing a positron emission tomography (PET) scan on the subject to detect the radiolabel in the subject. In some embodiments, a level and/or location of the radiolabel in the subject is determined. For example, the presence and/or amount of radiolabel in one or more different tissues of the subject are determined.

In some embodiments, the method comprises administering to a subject a first dose of a thymidine analog having a radiolabel and performing a PET scan on the subject to detect a level of and/or a location of radiolabel in the subject, administering a therapeutic agent (e.g., fusidic acid) to the subject, and administering to the subject a second dose of a thymidine analog having a radiolabel and performing a PET scan on the subject to detect a level of and/or a location of radiolabel in the subject.

Also provided herein are methods of risk stratification of subjects, for example, those known to have been exposed to a polyomavirus, such as JCV or BKV. In some embodiments, these methods comprises administering to a subject (e.g., a subject known to have been exposed to JCV or BKV, and/or suspected of having JCV or BKV) a thymidine kinase substrate having a label and detecting presence or absence of the label in at least one high risk tissue of the subject, wherein presence of the label in the high risk tissue of the subject above a reference level is indicative of presence of a polyomavirus in the high risk tissue of the subject and is indicative that the subject is at high risk for developing progressive multifocal leukoencephalopathy (PML). Non-limiting examples of “high risk” tissues include kidney, liver, bone marrow, and brain. Additional embodiments provide a method of reducing the risk of PML comprising withholding treatment with agents associated with PML if the method of risk stratification described above indicates that the subject is at high risk for developing PML.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 shows a graph of TK1 mRNA relative abundance values reported as reads per kilobase of transcript per million reads mapped (RPKM) in uninfected human cells and JCV-infected human cells. Relative abundance values of TK1 mRNA increased with JCV infection in human kidney cells (HuK(i)G10) and in human primary astrocytes (huAstrocyte). RNA sequencing of HuK(i)G10 and huAstrocyte cells was performed 8 days after infection with either archetype JCV virus or prototype PML virus.

FIG. 2 shows images of TK1 protein detected by immunofluorescence in uninfected HuK(i)G10 cells and JCV-infected HuK(i)G10 cells. Immunofluorescence staining of TK1 in uninfected and JCV-infected HuK(i)G10 cells was performed 7 days after infection with archetype JCV. Increased levels of TK1 protein were detected in JCV-infected HuK(i)G10 cells compared to uninfected HuK(i)G10 cells.

FIG. 3 shows images of TK1 protein detected by immunohistochemical staining in human brain tissue sections co-stained for T antigen. FIG. 3A shows that higher levels of TK1 protein were detected in the human brain PML lesion than in the control human brain. FIG. 3B shows that increased levels of TK1 protein and low levels of JCV viral particle (VP1) were detected in a human brain PML lesion. This shows that TK1 is induced in cells expressing JCV T antigen and is low in VP1 expressing cells. T antigen mediates entry into S phase.

FIG. 4 shows a graph of ³H-FLT uptake in uninfected and JCV-infected astrocytes. ³H-FLT uptake was higher in JCV-infected astrocytes as compared to uninfected astrocytes.

### DETAILED DESCRIPTION

Provided herein are methods for detecting polyomaviruses, which are useful, for example, for early detection of JCV and prevention of progressive multifocal leukoencephalopathy (PML).

Instead of encoding viral proteins that execute DNA replication, JCV encodes the early stage genes (large T antigen, small T antigen, and T′ antigen) that cause an infected cell to enter S (synthesis) phase of the cell cycle. The polyomavirus is then replicated by a cellular DNA polymerase complex of the host in concert with the JCV large T antigen, which has DNA binding and DNA helicase activities.

In polyomavirus infected cells, elevated levels of TK1, which phosphorylates thymidine transported across the cell membrane for use in DNA synthesis, enable efficient polyomavirus replication. The ability to cause quiescent cells to enter S phase, thereby inducing viral and cellular DNA replication, may be a common mechanism utilized by polyomaviruses, papillomaviruses and adenoviruses. Viral reservoirs are anatomical sites (e.g., tissues) in which viruses (e.g., polyomaviruses) often accumulate and persist. For example, JCV often accumulates in kidney, liver, bone marrow, and brain. Early detection of JCV reservoirs, for example, in particular “at-risk” regions, such as the brain, may help prevent and/or indicate a risk of the onset or progression of PML in immunocompromised patients. Prior to the present disclosure, however, detection of a polyomavirus reservoir was not performed.

Detecting a polyomavirus reservoir, as provided herein, relies, in part, on elevated levels of TK1 in cells infected with a polyomavirus. In some embodiments, methods of detecting a polyomavirus (e.g., JCV and BKV) in at least one tissue of a subject comprises administering to the subject a thymidine kinase substrate having a label and detecting presence or absence of the label in at least one tissue of the subject, wherein presence of the label above a baseline level is indicative of presence of a polyomavirus in the at least one tissue of the subject.

It should be understood that the term “indicative of presence of a polyomavirus” does not necessarily mean direct evidence that a polyomavirus is present in the tissue. The methods of the present disclosure rely on a detected intracellular level of a label that is attached to a thymidine kinase substrate. The labeled thymidine kinase substrate (e.g., FLT) is phosphorylated by thymidine kinase (e.g., thymidine kinase-1, TK-1). JCV-infected cells, as discussed herein, have higher TK-1 activity relative to most non-infected cells. In some embodiments, a labeled thymidine kinase substrate that is useful according to methods described herein is a substrate that is selectively retained inside a cell upon phosphorylation by thymidine kinase. In some embodiments, a phosphorylated thymidine kinase substrate is not incorporated into nucleic acid or other intracellular molecules, but has reduced cell membrane permeability relative to the unphosphorylated substrate, resulting in increased retention in a cell having elevated levels of thymidine kinase activity. For example, in some embodiments, the phosphorylated form of the labeled thymidine kinase substrate (e.g., FLT monophosphate) is not be incorporated into DNA and is impermeable to the cell membrane, thus, it is metabolically trapped inside the cell. In some embodiments, a monophosphorylated thymidine kinase substrate can be further phosphorylated to form a di- or tri- or other polyphosphorylated product that is retained in the cell (e.g., without being incorporated into a nucleic acid or other intracellular molecule). However, in some embodiments, a phosphorylated product (e.g., a mono-, di-, tri-, or poly-phosphorylated substrate also (or alternatively) can be incorporated into an intracellular molecule (e.g., a nucleic acid). The uptake and accumulation of the labeled thymidine kinase substrate (e.g., FLT monophosphate) can be used as an index of elevated TK-1 activity, which can be used as an index of the presence of JCV. Thus, the presence of the label (attached to a thymidine kinase substrate) above a reference level (e.g., in one or more tissues) “indicates” the (potential) presence of JCV but does not directly confirm that JCV is present. In some embodiments, one or more further studies may be conducted to directly confirm the presence of JCV in the subject.

A “thymidine kinase substrate” refers to a molecule that is phosphorylated by thymidine kinase to produce a phosphorylated thymidine kinase substrate. In some embodiments, the phosphorylated thymidine kinase substrate is further phosphorylated to produce diphosphorylated thymidine kinase and triphosphorylated thymidine kinase.

A “thymidine kinase substrate having a label” (a labeled thymidine kinase substrate) refers to a thymidine kinase substrate that comprises (e.g., is covalently linked to) a detectable label (a label that can be visualized or otherwise imaged or identified). In some embodiments, the label is a radiolabel. In some embodiments, the radiolabel is a positron emitter, such as ¹⁸F, or single photon emitting agent, such as ^{99m}Tc. Other PET/SPECT agents include ¹¹C, ¹⁸F, ⁶¹Cu, ⁶⁴Cu, ⁶⁷Cu, ⁶⁷Ga, ⁶⁸Ga, ⁷⁵Br, ⁷⁶Br, ⁹⁴mTc, ¹¹¹In, ¹²³I, ¹²⁴I, ¹²⁵I, ¹³¹I, ²⁰¹TI. (Aboagye, E. et al. WO 2010/023457. Nucleoside Analogues Useful as Positron Emission Tomography (PET) Imaging Agents).

In some embodiments, a labeled thymidine kinase substrate is a labeled thymidine. In some embodiments, a labeled thymidine kinase substrate is a labeled thymidine analog. In some embodiments, a labeled thymidine analog comprises a modified ribose, for example, 3′-deoxy-3′-[¹⁸F]-fluorothymidine (FLT). In some embodiments, a labeled thymidine analog comprises a modified thymine, for example, N³-[¹⁸F]fluoroethyl thymidine (FET). In some embodiments, a labeled thymidine analog comprises a modified ribose and a modified thymine. Examples of labeled thymidine analogs comprising a modified ribose and a modified thymine include, but are not limited to, 2‘-deoxy’-¹⁸F-fluoro-5-methyl-1-β-D-arabinofuranosyluracil (FMAU) and 2′-deoxy-2′-¹⁸F-fluoro-5-iodo-1-β-D-arabinofuranosyluracil (FIAU).

A labeled thymidine kinase substrate, as described herein, encompasses a thymidine kinase substrate that has been phosphorylated by TK1. For example, FLT is transported into the cell and is phosphorylated by TK1 into FLT monophosphate (FLT-MP). As discussed above, FLT monophosphate is impermeable to the cell membrane and, thus, is metabolically trapped inside the cell. In some embodiments, the labeled thymidine kinase substrate becomes incorporated into a labeled product (resulting from the action of the thymidine kinase on the substrate). In some embodiments, the labeled product can be a mono-, di-, tri-, or other poly-phosphorylated thymidine kinase substrate or other molecule into which the phosphorylated thymidine kinase substrate is incorporated. FLT phosphates are radiolabeled, impermeable to the cell membrane, and resistant to catabolism by thymidine phosphorylase in vivo.

“Administering” refers to implanting, absorbing, ingesting, injecting, inhaling, or otherwise introducing in a subject a thymidine kinase substrate having a label as described herein, or a composition thereof. A labeled thymidine kinase substrate may be administered orally or by a parenteral route, in the form of a pharmaceutical composition comprising the labeled thymidine kinase substrate, optionally in the form of a salt, in a pharmaceutically acceptable dosage form. Depending upon the route of administration, the compositions may be administered at varying doses. In some embodiments, the labeled thymidine kinase substrate may be administered orally in the form of tablets, capsules, ovules, elixirs, solutions, or suspensions. In some embodiments, the labeled thymidine kinase substrate may be administered parenterally, for example, intravenously, intra-arterially, intraperitoneally, intrathecally, intraventricularly, intrasternally, intracranially, intramuscularly, or subcutaneously.

A “subject” refers to a mammal being assessed for presence or absence of a polyomavirus with a method provided herein. A subject may be human, but also include other mammals, for example, those mammals useful as laboratory models for human disease (e.g., mouse, rat, rabbit, or dog). In some embodiments, a subject has been exposed to JCV or is known to have been exposed to JCV (e.g., have associated with or been in contact with someone having JCV). In some embodiments, exposure to JCV is indicated by detection of anti-JCV antibodies in a bodily fluid (e.g., serum) or the detection or JCV DNA in a bodily fluid (e.g., urine). In some embodiments, a subject has a weakened immune system or is otherwise immunocompromised. In some embodiments, a subject has an autoimmune disease, such as multiple sclerosis or Crohn's disease. In some embodiments, a subject is undergoing treatment for an autoimmune disease, such as multiple sclerosis or Crohn's disease. For example, a subject may be undergoing treatment with interferon beta-1b (BETASERON®, EXTAVIA®), interferon beta-la (AVONEX®), interferon beta-la (REBIF®), glatiramer acetate (COPAXONE®), natalizumab (TYSABRI®), dimethlyfumarate (TECIDERA®), mitoxantrone (NOVANTRONE®), fingolimod (GILENYA®), teriflunomide (AUBAGIO®), rituximab (MABTHERA®), alemtuzumab (MABCAMPATH®), daclizumab beta (ZINBRYTA®), and/or ocrelizumab (OCREVUS®). In some embodiments, a subject is between the age of 20 and 50 years. In some embodiments, a subject is between the age of 20 and 50 years and has an autoimmune disease, such as multiple sclerosis or Crohn's disease. In other embodiments, a subject is at least 50 years old and is undergoing treatment with natalizumab, rituximab, or alemtuzumab (or other immunomodulatory therapy). For example, a subject being tested for JCV (and, e.g., risk of developing PML) may be between the ages of 60 and 90 years and undergoing treatment with natalizumab, rituximab, or alemtuzumab (or other immunomodulatory therapy).

Methods of detecting a label, including a radiolabel, are known, and any of these methods may be used in accordance with the present disclosure. “Detecting” refers to the process of identifying the presence (existence of) or absence of (non-existence of) a label. In some embodiments, detecting a radiolabel by imaging of the radiolabel includes positron emission tomography (PET) imaging (Bailey, D. L et al. (2005). Positron Emission Tomography: Basic Sciences. Secaucus, N.J.: Springer-Verlag).

In some embodiments, detecting a radiolabel comprises single-photon emission computed tomography (SPECT) imaging. SPECT imaging provides less expensive but lower spatial resolution images than PET imaging. Examples of suitable PET/SPECT isotopes for labeling of a thymidine kinase substrate include ¹¹C, ¹⁸F, ⁶¹Cu, ⁶⁴Cu, ⁶⁷Cu, ⁶⁷Ga, ⁶⁸Ga, ⁷⁵Br, ⁷⁶Br, ⁹⁴mTc, ¹¹¹In, ¹²³I, ¹²⁴I, ¹²⁵I, ¹³¹I, ²⁰¹TI. (Aboagye, E. et al. WO 2010/023457. Nucleoside Analogues Useful as Positron Emission Tomography (PET) Imaging Agents).

Methods of the present disclosure may be used to detect a label in at least one tissue of a subject. A “tissue” refers to both localized and disseminated cell populations including an organ or a non-organ. In some embodiments, the tissue may be a tissue fragment or portion of the tissue. Examples of a tissue include, but are not limited to, brain, heart, lung, liver, kidney, blood vessel, connective tissue, bone marrow, muscle, gland, lymph node, stomach, spinal cord, cerebrospinal fluid (CSF), blood (plasma and/or serum), blood cells and urine.

TK1 phosphorylates thymidine and accordingly is upregulated during DNA synthesis and, thus has been a widely used indicator of cellular proliferation. A “highly proliferative tissue” refers to a cell population having a high proportion of cells capable of cell growth and cell division. Examples of highly proliferative tissue include, but are not limited to, gastrointestinal tract, thymus, bone marrow, liver, blood vessels, epithelial cells and skin fibroblasts. A “low proliferative tissue” refers to a cell population having a low proportion of cells capable of cell growth and cell division. Examples of low proliferative tissue include, but are not limited to, heart, brain, bone and muscle.

Experimental data provide herein show that TK1 is upregulated in JCV infected cells. A “reservoir” refers to anatomical sites (e.g., tissues) in which viruses (e.g., polyomaviruses) often accumulate and persist. Clinically relevant cellular tissues in which JCV reservoirs may persist include, but are not limited to, kidney, liver, bone marrow and/or brain.

A detectable level of a label indicative of the presence of a polyomavirus (e.g., JCV) should be compared to a reference level to account for the detection of background (low level) signal and to account for the detection of highly proliferative tissue, which will accumulate high levels of the label independent of the presence of a polyomavirus. Reference levels of a label can be obtained by measuring the level of the label in an intra-patient positive control of a highly proliferative tissue (e.g., bone marrow) and/or an intra-patient negative control of a low proliferative tissue (e.g., muscle). Reference levels can also be obtained from tissues of patients known not to have a polyomavirus. Reference levels used for PET scans, for example, are known in the art and may be used in accordance with the presence disclosure.

In some embodiments, a detectable level of a label (above the reference level) in a low proliferative tissue is indicative of the presence of a polyomavirus (e.g., JCV).

In some embodiments, a detectable level of a label is at least 10% above a reference level (e.g., level of label in a low-proliferative tissue in the subject). In some embodiments, a detectable level of a label is at least 15%, 20%, 25%, 30%, 35%, 40%, 45%, 50%, 55%, 60%, 65%, 70%, 75%, 80%, 85%, 90%, 95% or 100% above a reference level. In some embodiments, a detectable level of a label is 10-100%, 10-90%, 10-80%, 10-70%, 10-60%, 10-50%, 10-40%, 10-30%, 10-20%, 25-100%, 25-90%, 25-80%, 25-70%, 25-60%, 25-50%, 25-40%, 25-30%, 50-100%, 50-90%, 50-80%, 50-70% or 50-60% above a reference level.

In some embodiments, JCV detection in a clinically relevant site, such as the brain, may be used to assess the degree of risk of a subject for a JCV-related disease, such as progressive multifocal leukoencephalopathy (PML), which is a debilitating and often fatal demyelinating disease caused by JCV infection of oligodendrocytes in the brain.

Detecting a polyomavirus reservoir in a clinically relevant site of a subject that is typically a site of low proliferation (e.g., brain) may provide evidence for initiating treatment in a subject presenting relatively low viral levels in blood or urine. For example, relatively low levels of JCV may be detected in the urine of a subject suggesting that the subject is not at risk of developing PML. Nonetheless, a method, as provided herein, may be used to detect the presence of JCV accumulated in the brain of the subject, a clinically relevant cellular compartment affected by JCV. Presence of a JCV reservoir in the brain may suggest that the subject is at risk for developing PML and may benefit from undergoing PML treatment (or discontinuing/changing therapy that places a patient having JCV at risk of PML) before becoming symptomatic.

Detecting a polyomavirus in at least one tissue of a subject may comprise detecting a polyomavirus in one or multiple tissues of the subject. For example, a polyomavirus may be detected in at least 1, at least 2, at least 3, at least 4, at least 5, or at least 10 tissues of a subject. In some embodiments, a polyomavirus may be detected in 1, 2, 3, 4, 5, 6, 7, 8, 9, or 10 tissues of a subject. In some embodiments, a polyomavirus may be detected in 0-2 tissues, 0-4 tissues, 0-6 tissues, 0-8 tissues, or 0-10 tissues. In some embodiments, a polyomavirus may be detected in 2-10 tissues, 4-10 tissues, 6-10 tissues, or 8-10 tissues.

Methods described herein may be used to detect a polyomavirus in a subject include, but are not limited to, Polyomavirus BK (BKV), John Cunningham Polyomavirus (JCV), Karolinska Institute Polyomavirus (KIV), Washington University Polyomavirus (WUV), Merkel Cell Carcinoma-Associated Polyomavirus (MCV), Human Polyomavirus-6 (HPyV6), Human Polyomavirus-7 (HPyV7), Trichodysplasia Spinulosa-Associated Polyomavirus (TSV), Human Polyomavirus-9 (HPyV9), and MW Polyomavirus (MWPyV).

In some embodiments, a method of detecting a polyomavirus in at least one tissue of a subject who is immunocompromised, comprises administering to the subject who is immunocompromised a thymidine kinase substrate having a label and detecting presence or absence of the label in at least one tissue of the subject who is immunocompromised, wherein presence of the label above a reference level is indicative of presence of a polyomavirus in the at least one tissue of the subject who is immunocompromised.

In some embodiments, a method of detecting a polyomavirus in at least one tissue of a subject having Multiple Sclerosis, comprises administering to the subject having Multiple Sclerosis a thymidine kinase substrate having a label and detecting presence or absence of the label in at least one tissue of the subject having Multiple Sclerosis, wherein presence of the label above a reference level is indicative of presence of a polyomavirus in the at least one tissue of the subject having Multiple Sclerosis.

In some embodiments, a method of detecting a polyomavirus in at least one tissue of a subject having Crohn's Disease, comprises administering to the subject having Crohn's Disease a thymidine kinase substrate having a label and detecting presence or absence of the label in at least one tissue of the subject having Crohn's Disease, wherein presence of the label above a reference level is indicative of presence of a polyomavirus in the at least one tissue of the subject having Crohn's Disease.

In some embodiments, a method of detecting a polyomavirus in at least one tissue of a subject undergoing natalizumab therapy, comprises administering to the subject undergoing natalizumab therapy a thymidine kinase substrate having a label and detecting presence or absence of the label in at least one tissue of the subject undergoing natalizumab therapy, wherein presence of the label above a reference level is indicative of presence of a polyomavirus in the at least one tissue of the subject undergoing natalizumab therapy.

A subject may have, be at risk for, or be suspected of having a polyomavirus. A subject having a polyomavirus can be identified by a method disclosed herein or by routine medical examination (e.g., laboratory tests, cognitive tests, and/or behavior tests). A subject at risk for a polyomavirus can be a subject who is immunocompromised, for example, a subject having Multiple Sclerosis, Crohn's Disease, or undergoing natalizumab therapy. A subject suspected of having a polyomavirus might show one or more symptoms of the polyomavirus. A subject at risk for a polyomavirus can be a subject having one or more of the risk factors for that polyomavirus, for example, a subject undergoing natalizumab therapy is at risk for having JCV. In some instances, the human subject has, is suspected of having, or is at risk for a polyomavirus associated with immunodeficiency, for example, BKV and JCV.

A subject may have been treated, or is currently being treated for a polyomavirus. For example, a subject may have been treated or is currently being treated for JCV and may undergo a method described herein. A subject who has been treated for a polyomavirus can be a subject who was treated prior to undergoing the method described herein (e.g., two weeks, one month, one year, or more prior to undergoing the method as described herein) and may no longer be on the treatment at the time the method described herein is applied to the subject. A subject who has been treated for a polyomavirus can be a subject who was treated once, twice, or more prior to undergoing the method as described herein. A subject who is currently being treated for a polyomavirus is a subject who is still on such a treatment when the method described herein is applied to the subject.

Illnesses associated with polyomaviruses, such as JCV, typically occur in immunocompromised subjects, and thus the disclosed methods are particularly relevant to a subject who is immunocompromised. “Immunocompromised” refers to a subject having an immune system that is weakened or absent. Thus, a subject having a “weakened immune system” is immunocompromised. Subjects who have been infected with JCV and have an immune system that has been altered or weakened are believed to be at higher risk for PML. Possible factors that may lead to a weakened immune system include: AIDS, cancer (or its treatment), lupus and/or the treatments used to manage it, certain medications for autoimmune diseases, including multiple sclerosis, rheumatoid arthritis, psoriasis, Crohn's disease and related conditions, and certain medications used with organ transplantation. For subjects currently being treated with certain immunomodulatory therapies, prior use of other immunosuppressant drugs are believed to be a risk factor for PML development. A subject may have acquired immunodeficiency due to extrinsic factors including infections (e.g., human immunodeficiency virus (HIV)) or use of immunosuppressive therapies (e.g., natalizumab therapy). An immunocompromised subject may be a subject having an autoimmune disease, such as multiple sclerosis, or Crohn's disease, rheumatoid arthritis, psoriasis, or lupus. Subject being treated with certain medications used with organ transplants may also be immunocompromised, as indicated above. It has also been suggested that certain genetic factors in subjects may make them more likely to develop PML.

Any of the methods of detecting a polyomavirus in a subject, as provided herein, may be performed prior to initiating treatment with an immunosuppressive agent, during administration of an immunosuppressive agent, after an immunosuppressive agent has been administered, and/or after immunosuppressive treatment has been terminated.

It should be appreciated that any of the methods disclosed herein to detect a polyomavirus in a subject may be performed in combination with at least one additional assay to detect the presence or absence of polyomavirus in a subject.

Those of ordinary skill in the art are aware of numerous additional assays that are routinely utilized in the art to detect the presence or absence of polyomavirus nucleic acids (e.g., DNA) or polyomavirus protein in a urine sample, a cerebrospinal fluid sample, or a blood sample obtained from the subject. Assays for detecting nucleic acids from polyomavirus are well known in the art, and non-limiting examples include PCR, sequencing analysis, hybridization analysis, probe analysis and microarray analysis. Non-limiting examples of assays for the detection of polyomavirus polypeptides and/or antibodies include peptide sequencing analysis, mass spectrometry, immunosorbent assays (e.g., binding of the polypeptide to an antibody) and other protein arrays.

In some embodiments, the at least one additional assay comprises detecting the presence or absence of polyomavirus DNA or polyomavirus protein in a urine sample, a cerebrospinal fluid sample or a blood sample obtained from the subject. In some embodiments, the at least one additional assay comprises subjecting a urine sample (or other biological sample) obtained from the subject to a real-time polymerase chain reaction to detect polyomavirus DNA. In some embodiments, the at least one additional assay comprises detecting the presence or absence of an antibody that binds specifically to a polyomavirus in a serum sample obtained from the subject. The polyomavirus may be JCV or BKV, for example. In some embodiments, the at least one additional assay comprises staining a tissue sample obtained from the subject with an antibody that binds specifically to a polyomavirus (e.g., JCV or BKV).

In some embodiments, the at least one additional assay comprises determining a JCV antibody titer in a serum sample obtained from the subject and determining whether the JCV antibody titer is above, at, or below a pre-determined index level (e.g., 0.7, 0.8, 0.9, 1.0, 1.1, 1.2, 1.3, 1.4, or 1.5) (see, e.g., International Publication Number WO 2014/193804, incorporated herein by reference). In some embodiments, if the JCV antibody titer is above a pre-determined index level, e.g., 1.5, the subject is considered at higher risk of developing PML. In some embodiments, if the JCV antibody titer is at or below a pre-determined index level, e.g., 0.9, the subject is considered to be at lower risk of developing PML.

Additional assays to detect the presence or absence of a polyomavirus include but are not limited to histopathology, immunohistochemistry, flow cytometry, cytology, pathophysiological assays, including MRI and tomography, neurological assays biochemical assays. Other biochemical assays to detect the presence or absence of a polyomavirus include but are not limited to variant analysis, viral genome analysis, ELISA analysis, including the use of antibodies against one or more proteins of a polyomavirus, analysis of specific proteins, or platelet count.

It should be appreciated that any of the methods disclosed herein to detect a polyomavirus in a subject may be performed in combination with administering a therapeutic agent to treat a polyomavirus infection in the subject. As used herein, the term “treat” refers to the administration of a therapeutic agent to a subject, who is in need of the treatment, for example, having a polyomavirus, a symptom of the polyomavirus, or a predisposition toward the polyomavirus, with the purpose to cure, heal, alleviate, relieve, alter, remedy, ameliorate, improve, or affect the polyomavirus, the symptom of the polyomavirus, or the predisposition toward the polyomavirus. Conventional methods, known to those of ordinary skill in the art of medicine, can be used to administer the therapeutic agent (e.g., fusidic acid) to the subject, depending upon the polyomavirus to be treated or the site of the polyomavirus infection.

The disclosed methods performed in combination with administering a therapeutic agent, as used herein, embraces administering a first dose of a thymidine kinase substrate having a label to a subject and detecting the label in the subject, administering a therapeutic agent to the subject, and administering a second dose of the thymidine kinase substrate having a label and detecting the label in the subject. In some embodiments, the level and/or location of a label detected in a subject prior to administration of the therapeutic agent is compared to the level and/or location of a label detected in the subject post-administration of the therapeutic agent. In some embodiments, the disclosed method performed in combination with administering a therapeutic agent, as used herein, comprises administering a first dose of FLT and performing a PET scan on the subject, administering a therapeutic agent to the subject, and administering a second dose of FLT and performing a second PET scan on the subject.

In some embodiments, the disclosed method may be performed in combination with administering a therapeutic agent that inhibits DNA viral activity (e.g., JCV activity) to a subject. Exemplary therapeutic agents that inhibit DNA viral activity (e.g., JCV activity) include, but are not limited to chloroacetoxyquinoline, demethylnobiletin, propanil, aminoethoxydiphenylborane, 5-nitro-2-phenylpropylaminobenzoic acid, 3beta-hydroxyisoallospirost-9(11)-ene, leoidin, picropodophyllotoxin, thiabendazole, harmane, 6,4′-dihydroxyflavone, gentiopicroside, (R)angolensin, ptaeroxylin, dipyridamole, nabumetone, rosiglitazone, diltiazem hydrochloride, betamethasone, ichthynone, amcinonide, riluzole, flufenamic acid, chrysin, dictamnine, piplartine, peucenin, methoxyvone, isotretinoin, chloroxylenol, tomatine, primuletin, mefenamic acid, diethylstilbestrol, chloramphenicol palmitate, methylxanthoxylin, 1-alaninol, diclofenac sodium, flunixin, meglumine, dehydroabietamide, pachyrrhizin, dicumarol, diffractic acid, acemetacin, ginkgolic acid, xanthone, fusidic acid, polymyxin b sulfate, pyrantel pamoate, 4-(3-butoxy-4-methoxybenzyl)imidazolidin-2-one, miconazole nitrate, candesartan cilextil, endosulfan, dioxybenzone, tolfenamic acid, mefloquine, 2-methoxyxanthone, 3-hydroxy-4-(succin-2-yl)-caryolane delta-lactone, 5, 7-dihydroxyflavone, avocadanofuran, benzo(a)pyrene, beta-dihydrogedunol, decahydrogambogic acid, diosmetin, niloticin, pectolinarin, totarol acetate, 8-chloroadenosine, 3-deazaadenosine, 06-cyclohexylmethylguanine, 4-estren-3-beta 17-beta-diol 17-acetate, 5-beta-pregnan-3-alpha 6-alpha 20-beta-triol 20-acetate, or 4-pregnen-3-beta 20-beta-diol 20-acetate, or any combination thereof.

### EXAMPLES

### Example 1: TKJ mRNA Levels in Uninfected and JCV-Infected Human Cells

RNA extraction and quantification was performed to detect TK1 mRNA levels in uninfected and JCV-infected human kidney cells. Human kidney cells (HuK(i)G10) and primary human astrocytes (huAstrocyte) were plated, and the following day infected with JCV archetype virus from urine or prototype PML virus. Infected plates and uninfected control plates were harvested 8 days post-infection. RNA was isolated using the QIAGEN RNEASY® kit with DNase treatment to eliminate genomic DNA contamination. RNAs were quantified and polyadenylated transcripts amplified with random primers using a strand-specific technology compatible with the ILLUMINA® TruSeq Stranded mRNA High Sample Protocol and sequenced on the ILLUMINA® HiSeq 2500. The sequence reads were then mapped to the human genome to arrive at relative abundance values reported as reads per kilobase of transcript per million reads mapped (RPKM). Increased TK1 mRNA levels were detected in JCV-infected cells compared to uninfected cells (FIG. 1).

### Example 2: TK1 Protein Levels in Uninfected and JCV-Infected Human Kidney Cells

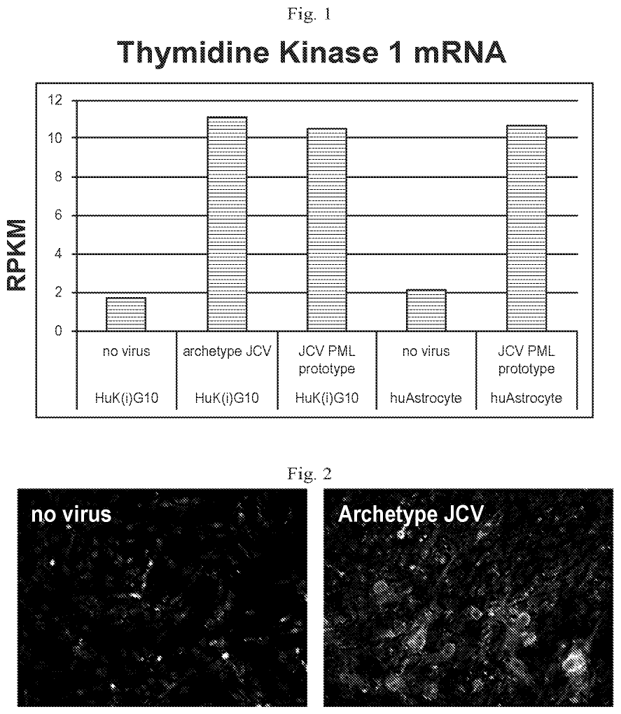

Immunofluorescent imaging was performed to detect TK1 protein levels in uninfected and JCV-infected human kidney cells. HuK(i)G10 cells were plated at a density of 8000 cells per well in a 96-well plate. After 24 hours, the media was removed and replaced with media containing archetype JCV virus. Seven days post infection, the cells were washed with 1× PBS and then fixed with 4% paraformaldehyde for 15 min. The following primary antibodies were used: PAB 2000 (Frisque R. et al. Virus Res. 1992 Sep. 15; 25(3):223-39) at a concentration of 1:1000 to detect expression of the viral early gene T-antigen, a human chimeric antibody of PAB597 (E. Harlow) at a concentration of 1:2000 to detect the expression of the viral late gene VP1, and FL-234 (Santa Cruz Biotechnology, catalog # sc-134475) at a concentration of 1:50 to detect thymidine kinase. The following secondary antibodies were used: anti-mouse ALEXA FLUOR® 594, anti-human ALEXA FLUOR® 647, and anti-rabbit ALEXA FLUOR® 488. The nucleus was stained with 4′,6-diamidino-2-phenylindole (DAPI) at a concentration of 1:10000. Images were acquired using the high content imaging platform Operetta (Perkin Elmer) and image analysis was done using Columbus software. Immunofluorescent images showed that significantly greater amounts of TK1 protein are present in JCV-infected cells compared to uninfected cells (FIG. 2).

### Example 3: Immunohistochemical Staining of TK1 in Human Brain Tissue

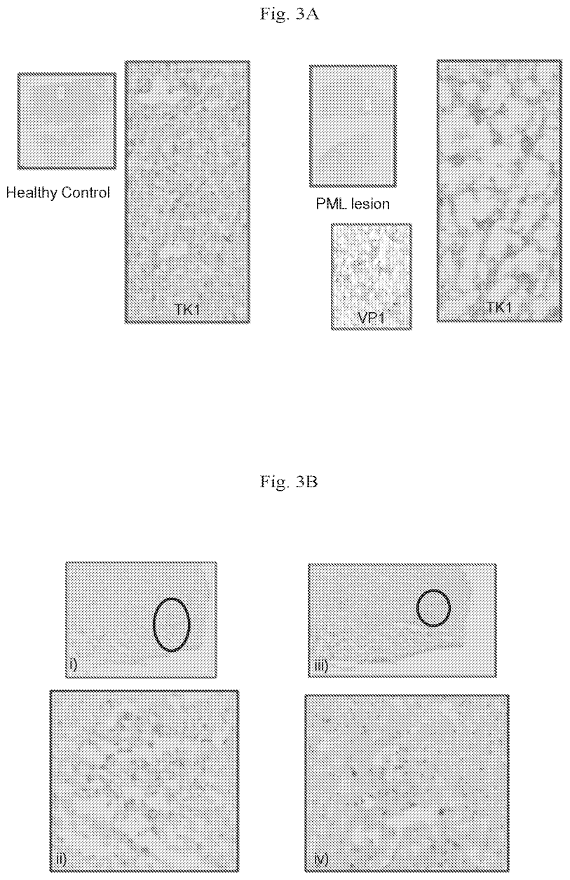

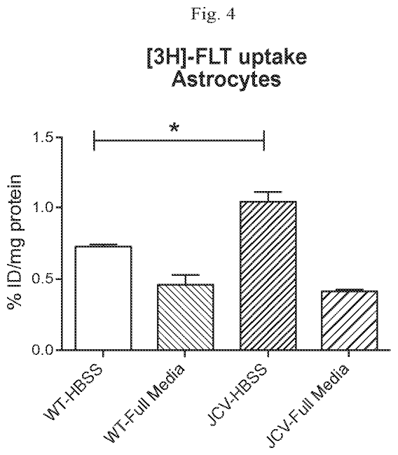

Levels of TK1 protein in normal healthy human brain tissue and brain tissue containing PML lesions was determined by immunohistochemical staining (FIGS. 3A and 3B). Staining of normal healthy brain tissue and brain tissue containing PML lesions showed that higher levels of TK1 protein are present in the PML lesion compared to the control tissue (FIG. 3). Furthermore, staining of PML lesions showed high levels of TK1 protein (FIG. 3A(i), green stain TK1, pink stain VP1; FIG. 3A(ii), zoomed in region of highlight area) in PML lesions having low VP1 (FIG. 3A(iii), pink stain Large T; FIG. 3A(iv) zoomed in region of highlighted area) levels.

### Example 4: H-FLT Uptake in Human Astrocytes

The activity of TK1 in uninfected and JCV-infected human astrocytes was determined by analyzing the cellular uptake of ³H-FLT, a radiolabeled TK1 substrate. JCV-infected human astrocytes showed elevated levels of TK1 activity, as determined by uptake of ³H-FLT, compared to uninfected cells (FIG. 5).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of detecting a polyomavirus in at least one tissue of a subject, the method comprising:
administering to the subject a thymidine kinase substrate having a label and detecting presence or absence of the label in at least one tissue of the subject, wherein presence of the label above a reference level is indicative of presence of a polyomavirus in the at least one tissue of the subject.

2. The method of claim 1, wherein the polyomavirus comprises John Cunningham virus (JCV).

3. The method of claim 1, wherein the polyomavirus comprises BK virus.

4. The method of any one of claims 1-3, wherein the thymidine kinase substrate having a label comprises a radiolabel.

5. The method of any one of claims 1-4, wherein the thymidine kinase substrate having a label comprises a thymidine analog.

6. The method of any one of claims 1-4, wherein the thymidine kinase substrate having a label is 3′-deoxy-3′-[¹⁸F]-fluorothymidine (FLT).

7. The method of any one of claims 1-6, wherein the label is detected by performing a positron emission tomography (PET) scan of the subject.

8. The method of claim 7, wherein the PET scan is performed on low proliferative tissue of the subject.

9. The method of claim 1 further comprising performing at least one additional assay to detect the presence or absence of polyomavirus in a subject.

10. The method of claim 9, wherein that at least one additional assay comprises detecting the presence or absence of polyomavirus DNA or polyomavirus protein in a urine sample, a cerebrospinal fluid sample or a blood sample obtained from the subject.

11. The method of claim 9, wherein the at least one additional assay comprises subjecting a urine sample obtained from the subject to a real-time polymerase chain reaction to detect polyomavirus DNA.

12. The method of claim 9, wherein the at least one additional assay comprises detecting the presence or absence of an antibody that binds specifically to a polyomavirus in a serum sample obtained from the subject.

13. The method of claim 9, wherein the at least one additional assay comprises staining a tissue sample obtained from the subject with an antibody that binds specifically to a polyomavirus.

14. The method of claim 12 or 13, wherein the polyomavirus is JCV.

15. The method of claim 12 or 13, wherein the polyomavirus is BKV.

16. The method of claim 9, wherein the at least one additional assay comprises determining a JCV antibody titer in a serum sample obtained from the subject.

17. The method of claim 16 further comprising determining whether the JCV antibody titer is above, at, or below a pre-determined index level.

18. The method of claim 17 further comprising determining that the subject is at a higher risk of developing PML if the JCV antibody titer is above a pre-determined index level, e.g., 1.5, or determining that the subject is at a lower risk of developing PML if the JCV antibody titer is at or below a pre-determined index level, e.g., 0.9.

19. The method of any one of claims 1-18, wherein the subject is known to have been exposed to a polyomavirus.

20. The method of any one of claims 1-19, wherein the subject has at least one risk factor for developing PML.

21. The method of claim 20, wherein the at least one risk factor is selected from a weakened immune system and genetic factors.

22. The method of claim 21, wherein the subject has acquired immune deficiency syndrome (AIDS), cancer, or an autoimmune disease.

23. The method of claim 22, wherein the autoimmune disease is selected from multiple sclerosis, Crohn's disease, rheumatoid arthritis, psoriasis, and lupus.

24. The method of any one of claims 1-23, wherein the subject is immunocompromised.

25. The method of claim 17 or 24, wherein the subject has multiple sclerosis.

26. The method of claim 17 or 24, wherein the subject has Crohn's disease.

27. The method of any one of claims 1-26, wherein the subject is undergoing natalizumab therapy.

28. A method of detecting John Cunningham virus (JCV) in a subject, comprising:
(a) performing at least one assay to detect the presence or absence of JCV in a subject; and
(b) administering to the subject a thymidine analog having a radiolabel and performing a positron emission tomography (PET) scan on the subject to detect the radiolabel in the subject.

29. The method of claim 28, wherein the thymidine analog having a radiolabel is 3′-deoxy-3′-[¹⁸F]-fluorothymidine (FLT).

30. The method of claim 28, wherein at least one assay performed in (a) comprises:
subjecting a urine sample obtained from the subject to a real-time polymerase chain reaction to detect JCV DNA; and/or
subjecting a cerebrospinal fluid sample or a blood sample obtained from the subject to an assay that uses an anti-JCV antibody to detect JCV protein.

31. A method, comprising:
(a) administering to a subject a first dose of a thymidine analog having a radiolabel and performing a positron emission tomography (PET) scan on the subject to detect a level of and/or a location of the radiolabel in the subject;
(b) administering a therapeutic agent to the subject; and
(c) administering to the subject a second dose of the thymidine analog having a radiolabel and performing a PET scan on the subject to detect a level of and/or a location of the radiolabel in the subject.

32. The method of claim 31, wherein the thymidine analog having a radiolabel is 3′-deoxy-3′-[¹⁸F]-fluorothymidine (FLT).

33. The method of claim 32, wherein the therapeutic agent is fusidic acid.

34. The method of any one of claims 31-33 further comprising comparing the level and/or location of radiolabel detected in (a) to the respective level and/or location of radiolabel detected in (c).

35. The method of any one of claims 31-34 further comprising
subjecting a urine sample obtained from the subject to a real-time polymerase chain reaction to detect JCV DNA; and/or
subjecting a cerebrospinal fluid sample or in a blood sample obtained from the subject to an assay that uses an anti-JCV antibody to detect JCV protein.

36. The method of any one of claims 28-35, wherein the subject is known to have been exposed to a polyomavirus.
